(19) United States
(12) Patent Application Publication
(10) Pub. No.: US 2005/0192244 A1
(43) Pub. Date: Sep. 1, 2005

(54) Histamine release inhibitor

(76) Inventors: Koichiro Komai, Kyoto-shi (JP); Tomoyuki Nakamura, Higashiyatsushiro-gun (JP); Yukihito Akiyama, Nirasaki-shi (JP); Ikuyo Kasuga, Kyoto-shi (JP); Kazumi Ueda, Daitou-shi (JP)

Correspondence Address: JACOBSON HOLMAN PLLC, 400 SEVENTH STREET N.W., SUITE 600, WASHINGTON, DC, 20004, US

(21) Appl. No.: 11/102,754

(22) Filed: Apr. 11, 2005

Related U.S. Application Data
(63) Continuation of application No. PCT/JP03/15708, filed on Dec. 9, 2003.

(30) Foreign Application Priority Data
Dec. 24, 2002 (JP) 2002-371680
May 12, 2003 (JP) 2003-132580
May 12, 2003 (JP) 2003-132594

(51) Int. Cl. A61K 31/7076

(52) U.S. Cl. 514/46; 514/47

(57) ABSTRACT

It was confirmed that the following <1> to <10> substances have an inhibitory activity on histamine release, whereby a histamine release inhibitor containing any of these substances as a major component could be obtained. <1> Adenosine <2> Guanosine <3> Cytidine <4> Uridine <5> Adenosine monophosphate (AMP) <6> Adenosine diphosphate (ADP) <7> Adenosine triphosphate (ATP)
<8> Adenosine derived from Phellinus linteus <9> N-hydroxy-N-methyl-adenosine
<10> N-hydroxy-N-methyl-adenosine derived from Phellinus linteus

This is a continuation of the Japanese PCT Application PCT/JP03/015708 filed Dec. 9, 2003 and published in Japanese.

### BACKGROUND OF THE INVENTION

1. Field of the Invention

The present invention relates to a histamine release inhibitor comprising a nucleoside derived from RNA (ribonucleic acid), such as adenosine, guanosine, cytidine or uridine.

In addition, the present invention relates to a histamine release inhibitor comprising an adenosine derivative, such as adenosine monophosphate (AMP, adenosine 5′-monophosphate), adenosine diphosphate (ADP, adenosine 5′-diphosphate), adenosine triphosphate (ATP, adenosine 5′-triphosphate), or N-hydroxy-N-methyl-adenosine.

2. Description of Related Art

Conventionally, the following substances are known as a substance having an inhibitory activity on histamine release.
<1> Tricaffeoylquinic acid and the like (JP-A-2002-80360)<
<2> Tomato extract (JP-A-2002-80387)<
<3> Apigenin and the like (JP-A-2000-86510)
<4> Bark extracts from Cinnamomum loureirii Nees, Myrica rubra Sieb and the like (JP-A-10-287582)

In addition, the present applicant has already filed a patent application (Japanese Patent Application No. 2002-335238) for a histamine release inhibitor comprising <5> a substance derived from Phellinus linteus mycelium as a substance having an inhibitory activity on histamine release.

However, an inhibitory activity on histamine release of nucleosides derived from RNA (adenosine, guanosine, cytidine and uridine) was not known.

In addition, an inhibitory activity on histamine release of adenosine derivatives (adenosine monophosphate, adenosine diphosphate, adenosine triphosphate and N-hydroxy-N-methyl-adenosine) was not known, either.

Therefore, the present inventors carried out an intensive investigation by focusing on the inhibitory activity on histamine release as a pharmacological effect of the nucleosides derived from RNA and the adenosine derivatives described above. As a result, they found that these substances have a significant inhibitory activity on histamine release, thus the present invention has been accomplished.

In addition, the inventors carried out an intensive investigation by focusing on the inhibitory activity on histamine release of Phellinus linteus mycelium. As a result, they found that N-hydroxy-N-methyl-adenosine extracted and isolated from Phellinus linteus mycelium has a significant inhibitory activity on histamine release, thus the present invention has been accomplished.

### SUMMARY OF THE INVENTION

The invention of this application includes the following (1) to (11) aspects.

(1) A histamine release inhibitor comprising a nucleoside derived from RNA.

(2) The histamine release inhibitor described in (1), wherein the nucleoside derived from RNA is adenosine.

(3) The histamine release inhibitor described in (2), wherein adenosine obtained from Phellinus linteus mycelium is used.

(4) The histamine release inhibitor described in (1), wherein the nucleoside derived from RNA is guanosine.

(5) The histamine release inhibitor described in (1), wherein the nucleoside derived from RNA is cytidine.

(6) The histamine release inhibitor described in (1), wherein the nucleoside derived from RNA is uridine.

(7) A histamine release inhibitor comprising adenosine monophosphate (AMP).

(8) A histamine release inhibitor comprising adenosine diphosphate (ADP).

(9) A histamine release inhibitor comprising adenosine triphosphate (ATP).

(10) A histamine release inhibitor comprising N-hydroxy-N-methyl-adenosine.

(11) The histamine release inhibitor described in (10), wherein N-hydroxy-N-methyl-adenosine obtained from Phellinus linteus mycelium is used.

The reason why the invention of this application includes the above-mentioned aspects is that, though nucleosides derived from RNA, namely, <1> adenosine, <2> guanosine, <3> cytidine, <4> uridine and the like have been conventionally known as a substance related to a nucleic acid, an inhibitory activity on histamine release of these substances was not known. In a similar way, the reason is that, though adenosine derivatives, namely, adenosine monophosphate (AMP), adenosine diphosphate (ADP), adenosine triphosphate (ATP) and the like have been conventionally known as a substance related to a nucleic acid, an inhibitory activity on histamine release of these substances was not known.

Further, the reason is that, though it was not known in the past that adenosine and N-hydroxy-N-methyl-adenosine extracted and isolated from Phellinus linteus mycelium have an inhibitory activity on histamine release, it was confirmed that they have a significant inhibitory activity on histamine release in the studies performed by the present inventors.

### BRIEF DESCRIPTION OF THE DRAWINGS

FIG. 1 is a diagram showing a fractionation process of hot water extract of Phellinus linteus mycelium.

FIG. 2 is a fraction chart of Fr7.

FIG. 3 is a chart showing the mass spectrum (GC-MS) of Compound A.

FIG. 4 is a chart showing the ¹H-NMR spectrum of Compound A.

FIG. 5 is a chart showing the ¹³C-NMR spectrum of Compound A.

FIG. 6 is a chart showing the infrared spectrum of Compound A.

FIG. 7 is a chart showing the ultraviolet and visible spectrum of Compound A.

FIG. 8 is a chart showing the mass spectrum (GC-MS) of Compound B.

FIG. 9 is a chart showing the ¹H-NMR spectrum of Compound B.

FIG. 10 is a chart showing the ¹³C-NMR spectrum of Compound B.

FIG. 11 is a chart showing the infrared spectrum of Compound B.

FIG. 12 is a chart showing the ultraviolet and visible spectrum of Compound A.

### DESCRIPTION OF THE PREFERRED EMBODIMENTS

<Confirmation of Inhibitory Effect on Histamine Release>

(1) With regard to the following substances, an inhibitory effect on type I allergic reaction was investigated by investigating an inhibitory effect on histamine release from mast cells.
<1> Adenosine
<2> Guanosine
<3> Cytidine
<4> Uridine
<5> Adenosine monophosphate (AMP)
<6> Adenosine diphosphate (ADP)
<7> Adenosine triphosphate (ATP)
<8> Adenosine derived from Phellinus linteus (described later as Compound A)
<9> N-hydroxy-N-methyl-adenosine derived from Phellinus linteus (described later as Compound B)
<10> A commercially available histamine inhibitor (brand name: Intal, major component: disodium cromoglycate, manufacturer: Fujisawa Pharmaceutical Co., Ltd.)

With regard to the foregoing <1> to <7> substances, commercially available reagents (manufactured by Wako Pure Chemical Industries, Ltd.) were used.

(2) Preparation Method of Test Animal and Cell

(a) For a test animal, a male Wister rat (at 6 to 8 weeks of age) was used.

(b) Preparation Method of Cell

After a rat was sacrificed by exsanguination under anesthesia with ether, 15 ml of Tyrode Buffer was injected into the peritoneal cavity of the rat and intraperitoneal cavity fluid was recovered. Ten ml of Tyrode Buffer was further injected into the peritoneal cavity of the rat. Intraperitoneal cavity fluid was further recovered and combined with the intraperitoneal fluid recovered before. This intraperitoneal fluid was centrifuged at 800 rpm for 10 minutes at 4° C. and the supernatant was removed and filtrated.

Further, cell suspension, to which Tyrode Buffer was added, was centrifuged at 800 rpm for 10 minutes at 4° C., the supernatant was removed and the cells were homogenized. This cell suspension was measured with an erythrocytometer, and prepared to be 8×10⁶ cells/ml for use.

(3) Experimental Method of Bioassay and Analytical Method of Histamine

(a) Experimental Method of Bioassay

The cell suspension (370 μl) dispensed into a 1.2 ml-microtube was pre-incubated in a water bath at 37° C.

Ten minutes later, 20 μl of sample prepared at each concentration was added.

Further, 10 minutes later, 10 μl of compound 48/80 (a mast cell stimulation agent; manufactured by Wako Pure Chemical Industries, Ltd.) (0.5 μg/ml), which is an allergy inducer, was added.

Fifteen minutes later, the reaction was terminated on ice.

Further, to remove the mast cells, centrifugation was carried out at 13,000 rpm for 15 minutes at 4° C. and the supernatant was recovered.

This supernatant was used as a sample for diazotation in HPLC analysis.

(b) Preparation of Diazotized Hitamine Derivative

An equal amount of 20 mM p-nitroaniline hydrochloric acid solution and 200 mM sodium nitrite aqueous solution were mixed well (diazo reagent).

To 10 μl of the diazo reagent, 20 μl of the foregoing sample for (histamine) diazotation was added and mixed well by using a vortex mixer.

Further, 30 μl of sodium carbonate ethanol solution was added and mixed well by using a vortex mixer.

This solution (20 μl) was used in HPLC analysis.

(c) The analysis of histamine was carried out by the diazotation method (Specific Determination of Histamine in Fish by High-performance Liquid Chromatography after Diazo Coupling: Biosci. Biotech. Biochem. 59 (7) 1208-1210 (1995)).

The conditions for HPLC analysis of histamine (diazotation method) in bioassay are shown in Table 1.

TABLE 1

| Item | Condition |
|---|---|
| Column | COSMOSIL 5C18-AR-II |
| | (150 mm/4.0 mm) |
| Temperature | 40° C. |
| Flow rate | 1 ml/min |
| Solvent | A: acetonitrile |
| | B: 5% acetonitrile + 0.1% trifluoroacetic acid |
| Condition of solvent | Ratio in Solvent B (%) |
| gradient | 0 min.: 20% |
| | 6 min.: 38% |
| | 13 min.: 20% |
| Analysis time | 20 min. |
| Detector and wavelength | Visible ultraviolet detector |
| | (460 nm) |

(4) The results of the inhibitory effects on histamine release from mast cells of a specified concentration of <1> adenosine, <2> guanosine, <3> cytidine, <4> uridine, <5> adenosine monophosphate (AMP), <6> adenosine diphosphate (ADP), <7> adenosine triphosphate (ATP) (<1> to <7> substances were from Wako Pure Chemical Industries, Ltd.), <8> adenosine derived from Phellinus linteus (described later as Compound A), <9> N-hydroxy-N-methyl-adenosine derived from Phellinus linteus (described later as Compound B), and <10> a commercially available histamine inhibitor (brand name: Intal, major component: sodium cromoglycate, manufacturer: Fujisawa Pharmaceutical Co., Ltd.) are shown in Table 2. The inhibition ratios shown in Table 2 are mean values of three measurement values in each case.

TABLE 2

| | | Inhibition |
|---|---|---|
| | Concentration | Ratio |
| Sample | (μg/ml) | (%) |
| Adenosine | 1 | 27.5 |
| Adenosine | 5 | 43.8 |
| Adenosine | 20 | 59.3 |
| Adenosine | 50 | 74.7 |
| Guanosine | 5 | 39.0 |
| Cytidine | 5 | 38.0 |
| Uridine | 5 | 34.0 |
| Adenosine monophosphate | 10 | 54.9 |
| Adenosine diphosphate | 10 | 47.8 |
| Adenosine triphosphate | 10 | 40.0 |
| Adenosine derived from Phellinus linteus | 5 | 41.5 |
| N-hydroxy-N-methyl-adenosine | 5 | 27.0 |
| Commercially available histamine inhibitor | 5 | 19.0 |
| (Intal) | | |
| Commercially available histamine inhibitor | 10 | 38.6 |
| (Intal) | | |

The value of inhibition ratio was calculated from the following calculation formula.

Calculation Formula:
(1−(peak integral value of sample)/(peak integral value of control))×100

Table 2 demonstrates that all of the foregoing <1> to <9> substances have an inhibitory effect on histamine release.

<Preparation Method of Adenosine Derived from Phellinus linteus and N-hydroxy-N-methyl-adenosine Derived from Phellinus linteus>

(a) Phellinus linteus Mycelium

By using a large-scale tank (1,000 L), mycelium of Phellinus linteus was seeded in a culture medium at a initial pH of 5.5 containing 4.0% glucose as a carbon source, 0.3% yeast extract and 0.3% polypeptone as a natural nitrogen source, 0.05% KH₂PO₄ and 0.05% Na₂HPO₄, and aeration was carried out by compulsorily passing sterile air which had been passed through a filter with a pore size of 0.22 μm through the culture medium, and culture was carried out at 28° C. for 45 days.

This culture solution was centrifuged and the obtained mycelium was lyophilized, thus dry powder of Phellinus linteus mycelium was obtained.

Phellinus linteus used in the invention of this application is a strain deposited as PL-08 strain at Applied Fungi Institute in IBI Co., Ltd. PL-08 strain was obtained by extracting and refining mycelium from the fruit body of Phellinus linteus, which was collected at Nishimorokata-gun Suki-son in Miyazaki-ken in October 1998. The strain used in the invention was the one determined <1> to have yellow-brown cystidia specific to the fruit body of Phellinus linteus and <2> to be identified as Phellinus linteus based on the basidiospore formation, which had been assayed by Dr. Yasuhisa Abe of Forest Biology Division, Forest Microbiology Section, Wood decay and Mycology Laboratory in Forestry and Forest Products Research Institute of Forestry Agency, the Ministry of Agriculture, Forestry and Fisheries of Japan.

(b) Hot Water Extract of Phellinus linteus Mycelium

To the obtained dry powder of Phellinus linteus mycelium, ion exchanged water in an amount of 10 times the dry powder was added, and hot water extraction treatment was carried out at 100° C. for 2 hours. Then, insoluble substances were removed, whereby a hot water extract of Phellinus linteus mycelium was obtained. This hot water extract was concentrated under vacuum at about 70° C.

From the foregoing 1300 g of dry powder of Phellinus linteus mycelium, about 280 g of the foregoing concentrate of hot water extract of Phellinus linteus was obtained.

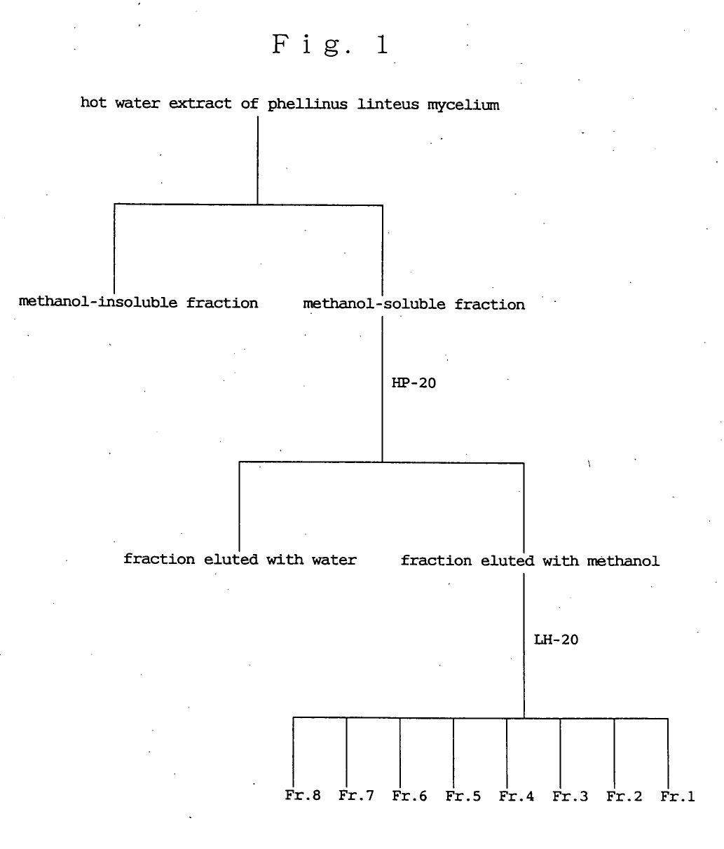

This hot water extract was fractionated by the method shown in FIG. 1.

(c) Fractionation of Hot Water Extract of Phellinus linteus Mycelium with Methanol

To the foregoing hot water extract of Phellinus linteus, methanol in an amount of 3 times the hot water extract was added, and the mixture was let stand for 2 hours. Then, the mixture was centrifuged, whereby a methanol-soluble fraction and a methanol-insoluble fraction were obtained.

From the foregoing 280 g of concentrate of hot water extract of Phellinus linteus, 51 g of methanol-soluble fraction and 226 g of methanol-insoluble fraction were obtained.

(d) Fractionation of Methanol-Soluble Fraction with Ion Adsorbent

To ion adsorbents (Diaion HP-20 (manufactured by Nippon Rensui Co.) hereinafter also referred to as HP-20) packed in a column, the foregoing methanol-soluble fraction was adsorbed, and fractionated into a fraction eluted with water and a fraction eluted with methanol.

From the foregoing 51 g of methanol-soluble fraction, 43 g of fraction eluted with water and 4.0 g of fraction eluted with methanol were obtained.

(e) Fractionation of Methanol-Soluble Fraction After Treatment with Ion Absorbent

By using Sephadex LH-20 (manufactured by Pharmacia, hereinafter also referred to as LH-20) column, 4.0 g of methanol-soluble fraction obtained in (c) described above was fractionated as follows by the gel filtration chromatography.

Elution of the target component was carried out by using distilled water, the entire solution was fractionated into 8 fractions (Fr1 to Fr8) in total (the foregoing 4.0 g of methanol-soluble fraction was fractionated into eight 0.5 g aliquots).

The conditions for fractionation with LH-20 are shown in Table 3.

TABLE 3

| Item | Condition |
|---|---|
| Column | Sephadex LH-20 fraction column |
| | (4.6 mm × 150 mm) |
| Temperature | 40° C. |
| Flow rate | 1 ml/min |
| Solvent | Distilled water |
| Analysis time | 30 to 40 min. |
| Detector and wavelength | Visible ultraviolet detector |
| | (259 nm) |

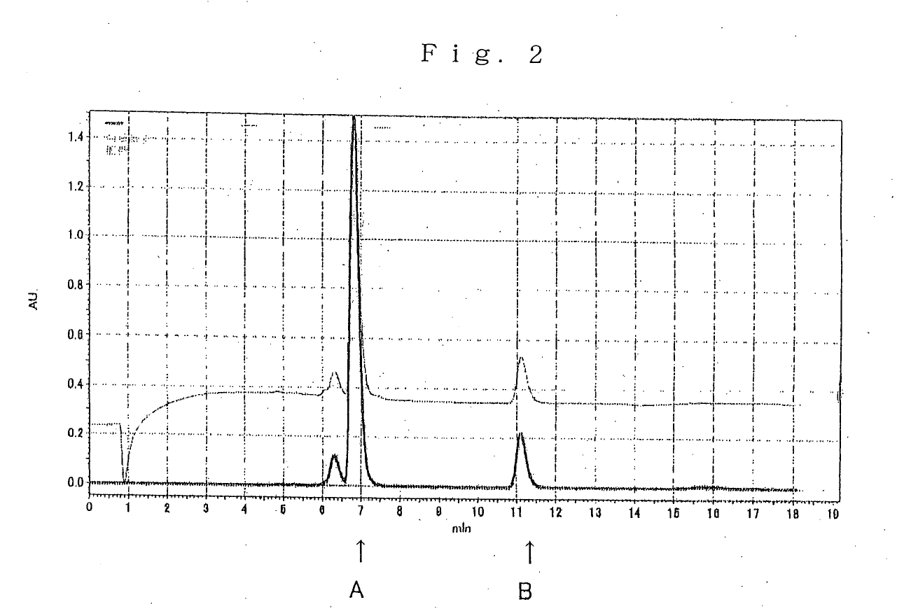

Among the obtained 8 fractions, with regard to Fr7 (see FIG. 1), Component A and Component B were isolated and fractionated by HPLC with an ODS column (filler material in which an octadecyl silyl group had been chemically bound to a silica gel support, manufactured by Simadzu Co). The conditions for HPLC analysis are shown in Table 4, and the fraction chart is shown in FIG. 2.

TABLE 4

| Item | Condition |
|---|---|
| Column | ODS fraction column |
| Temperature | 40° C. |
| Flow rate | 1 ml/min |
| Solvent | 40% methanol aqueous solution |
| Analysis time | 20 min. |
| Detector and wavelength | Visible ultraviolet detector |
| | (259 nm) |
| Fraction sample | Sample dissolved in 40% methanol aqueous |
| | solution |

(f) Identification of Component A

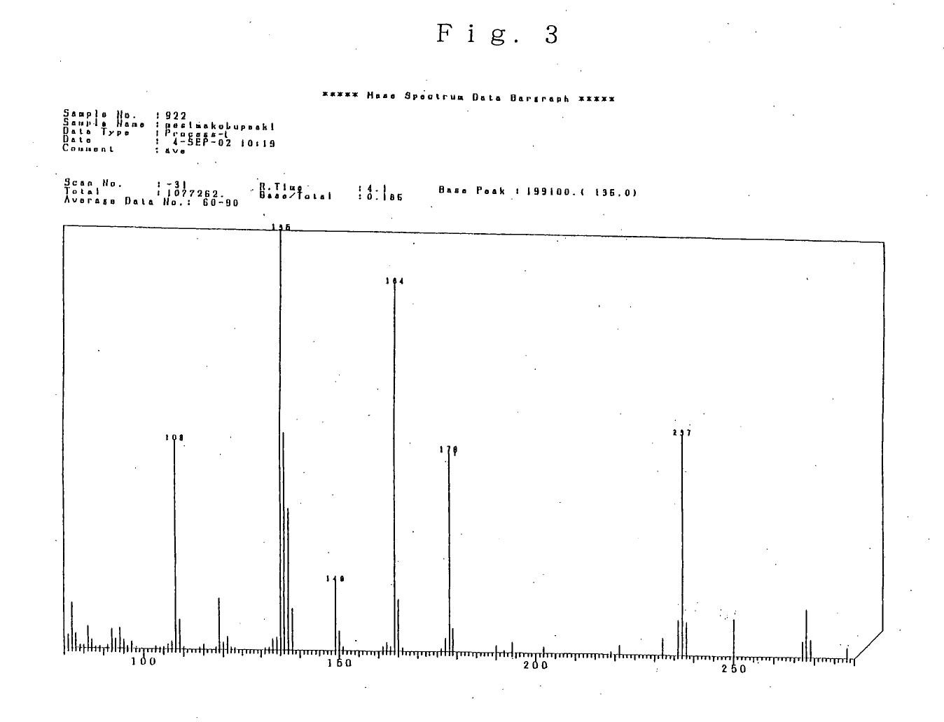

The Component A (methanol solution) isolated and fractionated by HPLC was subjected to mass spectroscopy (GC-MS), and the molecular weight of the compound associated with peak A (hereinafter referred to as Compound A) and a functional group bound to this Compound A were estimated. As a result, the molecular weight of Compound A was estimated to be 267. The spectrum chart is shown in FIG. 3.

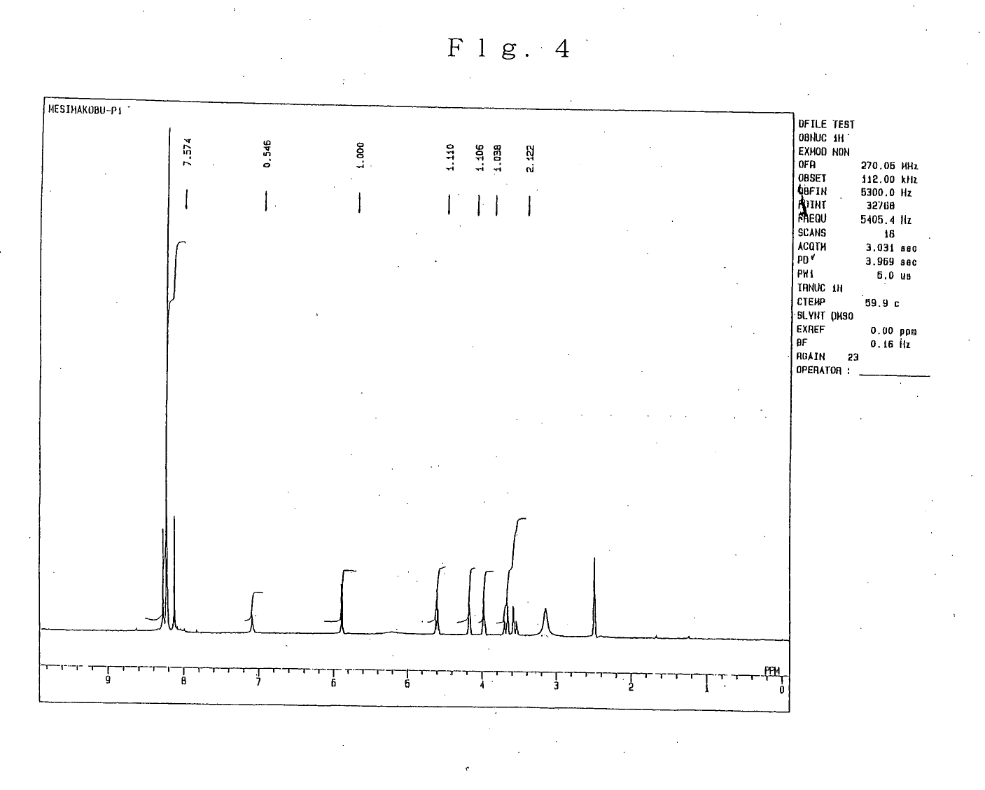

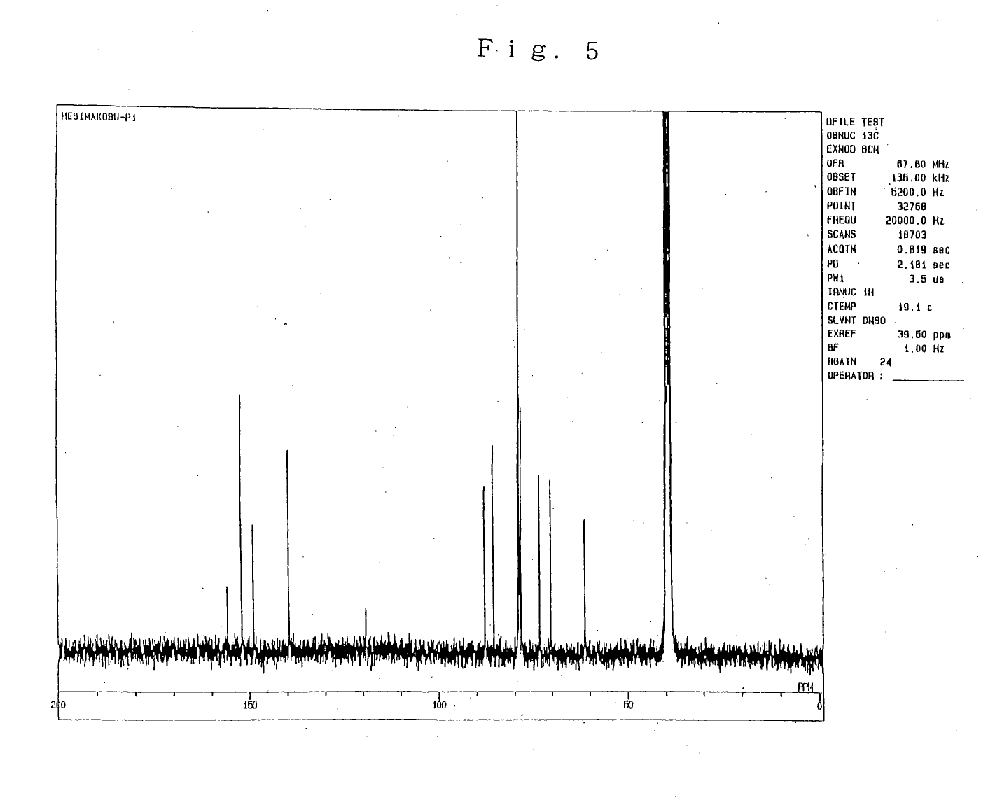

The Compound A (dissolved in DMSO containing several drops of chloroform) was subjected to nuclear magnetic resonance (¹H-NMR and ¹³C-NMR). The results are shown in FIG. 4 (¹H-NMR) and FIG. 5 (¹³C-NMR), respectively.

From FIG. 4, hydrogen signals were observed in the regions of a saccharide and an aromatic ring in Compound A. In addition, from FIG. 5, the number of carbon atoms in Compound A was estimated to be 10, and carbon signals were observed in the regions of a saccharide and an aromatic ring.

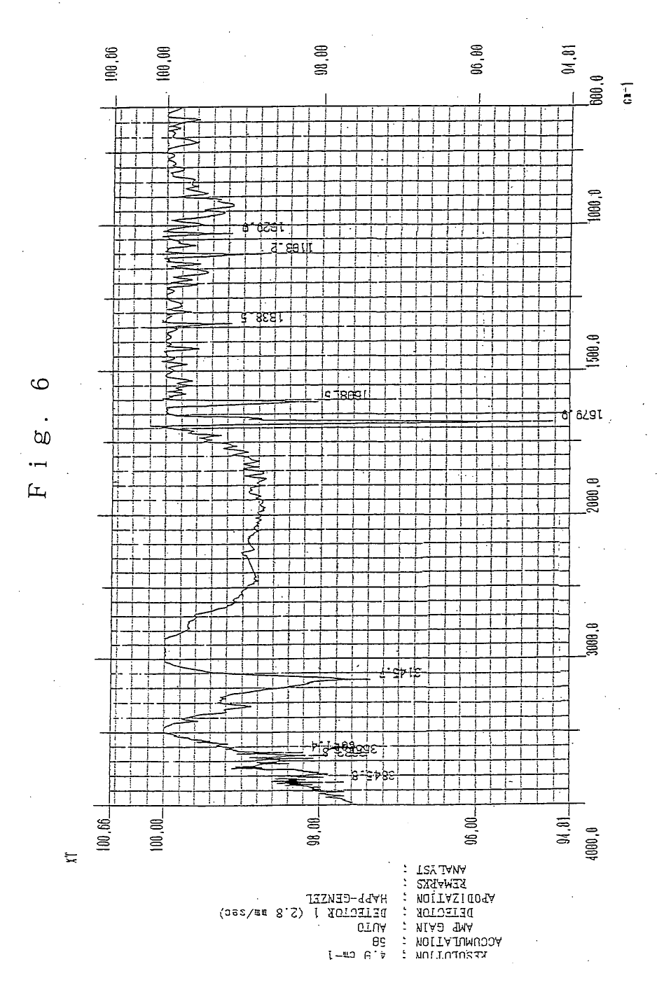

With regard to Compound A, its infrared spectrum was measured. The result is shown in FIG. 6.

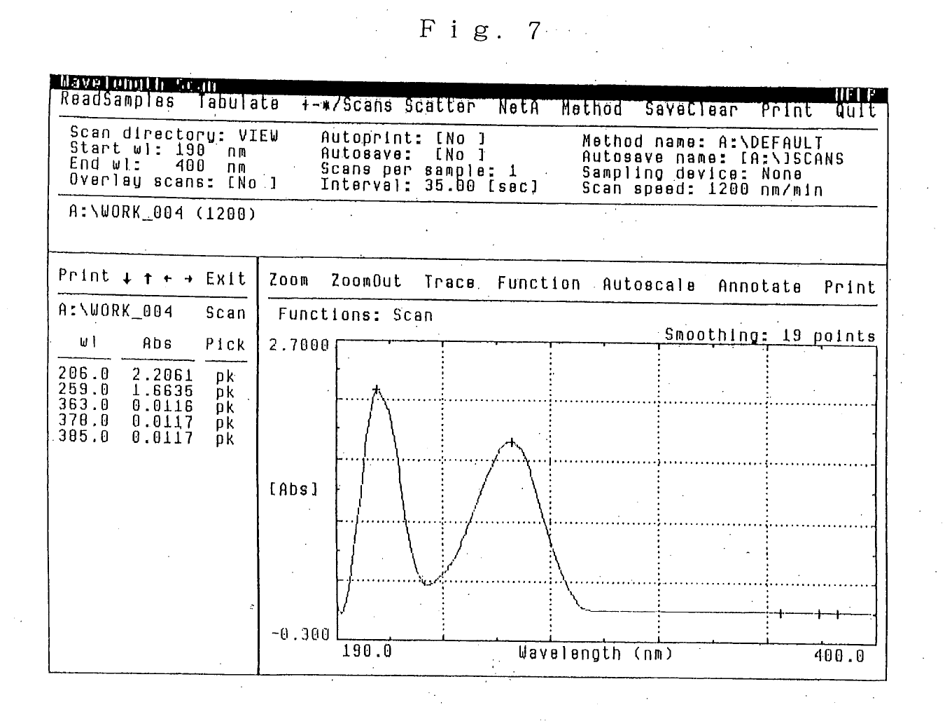

With regard to Compound A obtained by HPLC (dissolved in ethanol), its ultraviolet and visible spectrum was measured (λmax 259 nm). The result is shown in FIG. 7.

From the results described above, Compound A was identified as adenosine (the following formula).

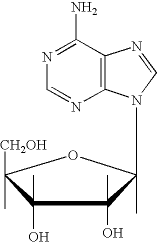

(g) Identification of Component B

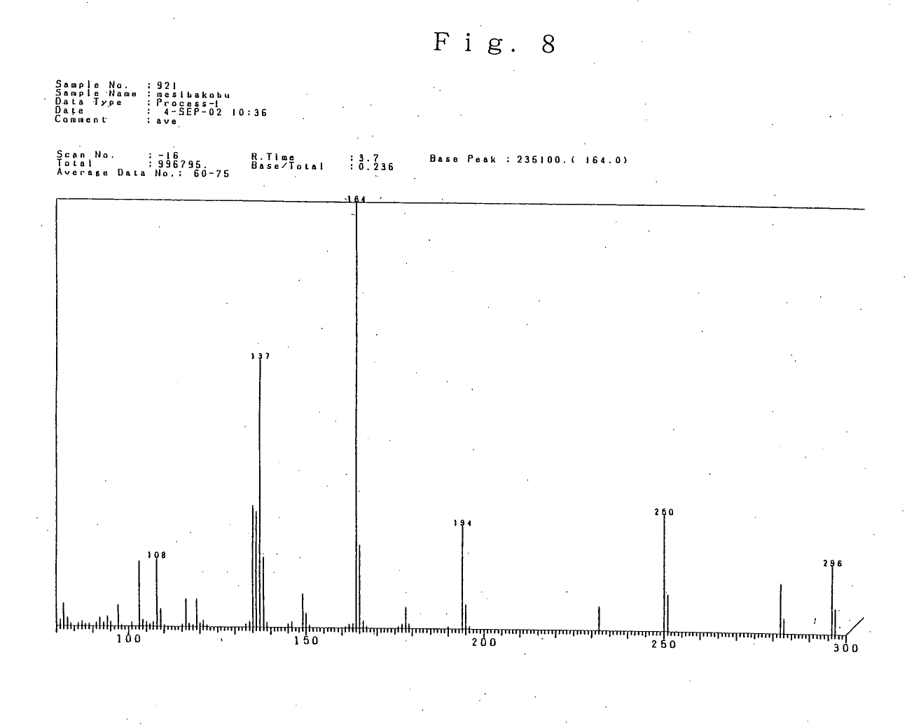

The Component B (methanol solution) isolated and fractionated by HPLC was subjected to mass spectroscopy (GC-MS), and the molecular weight of the compound associated with peak B (hereinafter referred to as Compound B) and a functional group bound to this Compound B were estimated. As a result, the molecular weight of Compound B was estimated to be 297. The spectrum chart is shown in FIG. 8.

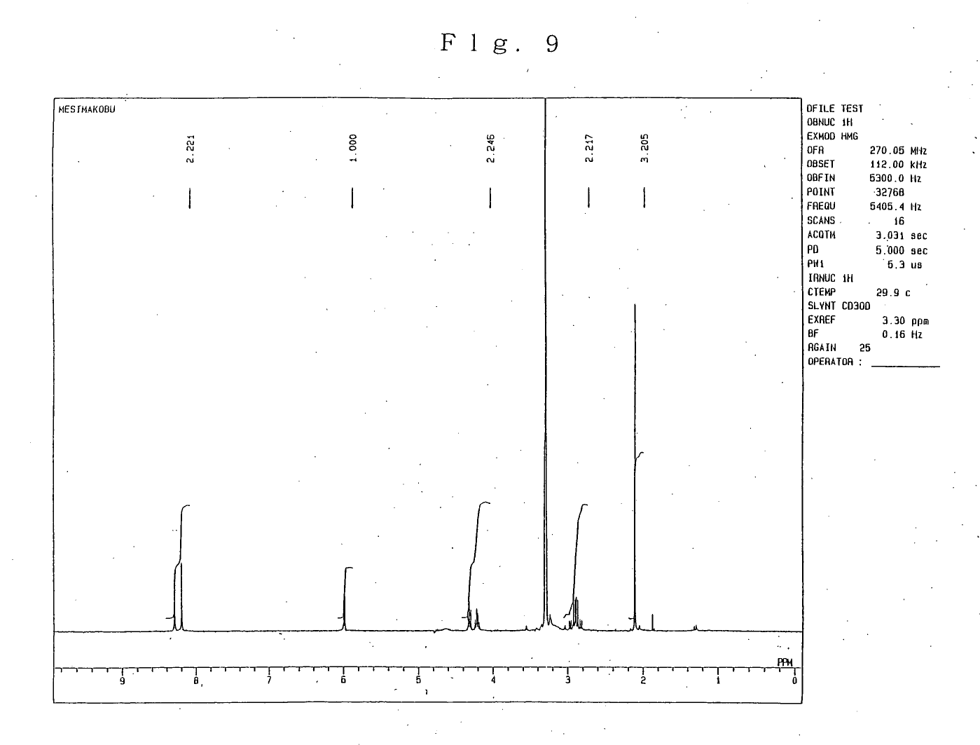

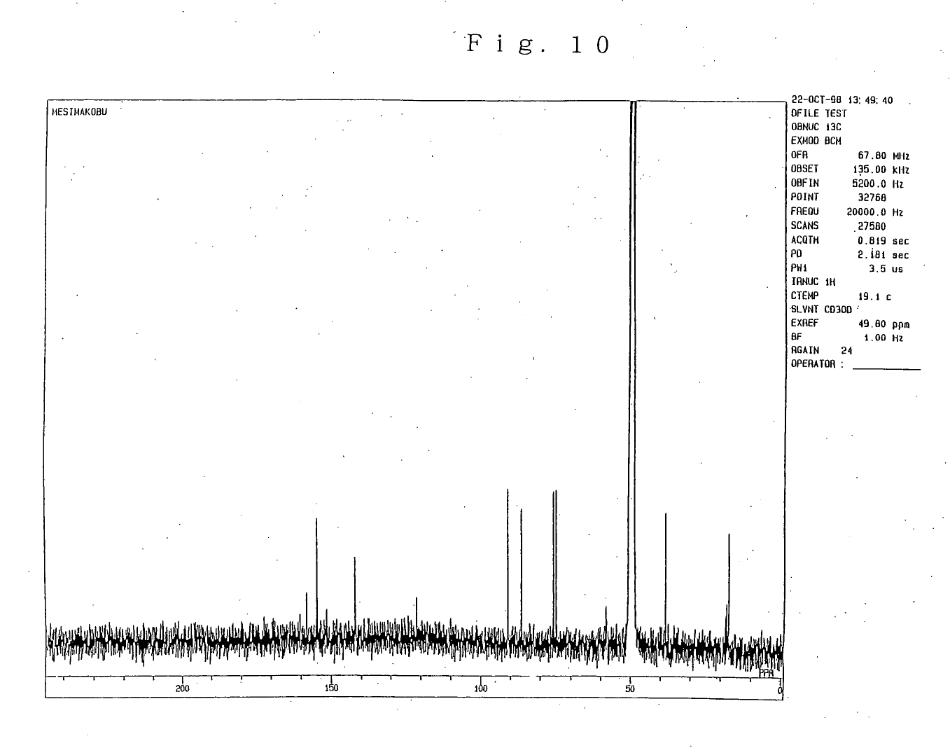

The Compound B (dissolved in DMSO containing several drops of chloroform) was subjected to nuclear magnetic resonance (¹H-NMR and ¹³C-NMR). The results are shown in FIG. 9 (¹H-NMR) and FIG. 10 (¹³C-NMR), respectively.

From FIG. 9, hydrogen signals were observed in the regions of a saccharide and an aromatic ring in Compound B. In addition, from FIG. 10, the number of carbon atoms in Compound B was estimated to be 11, and carbon signals were observed in the regions of a saccharide and an aromatic ring.

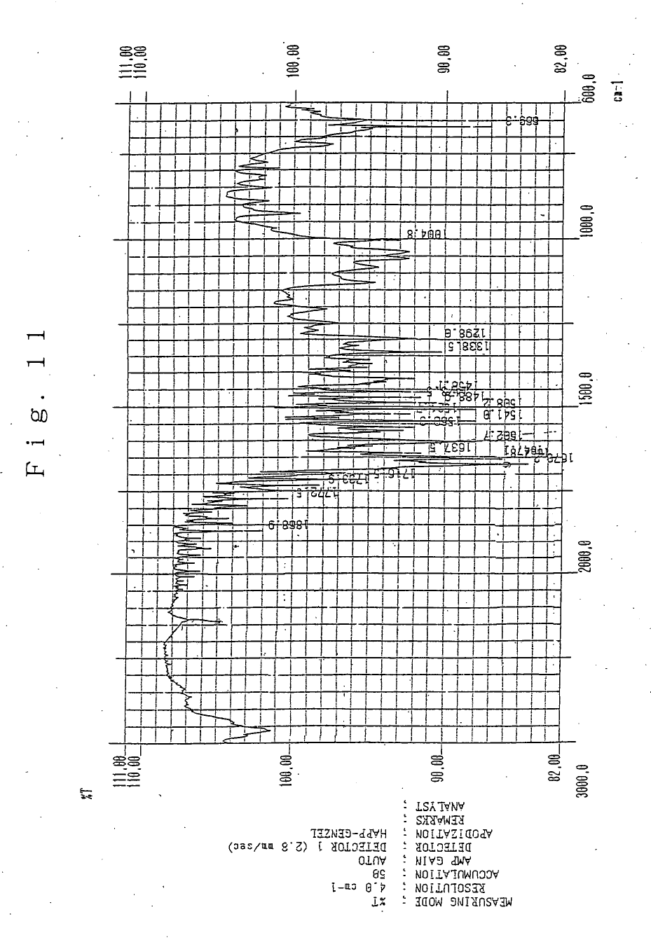

With regard to Compound B, its infrared spectrum was measured. The result is shown in FIG. 11.

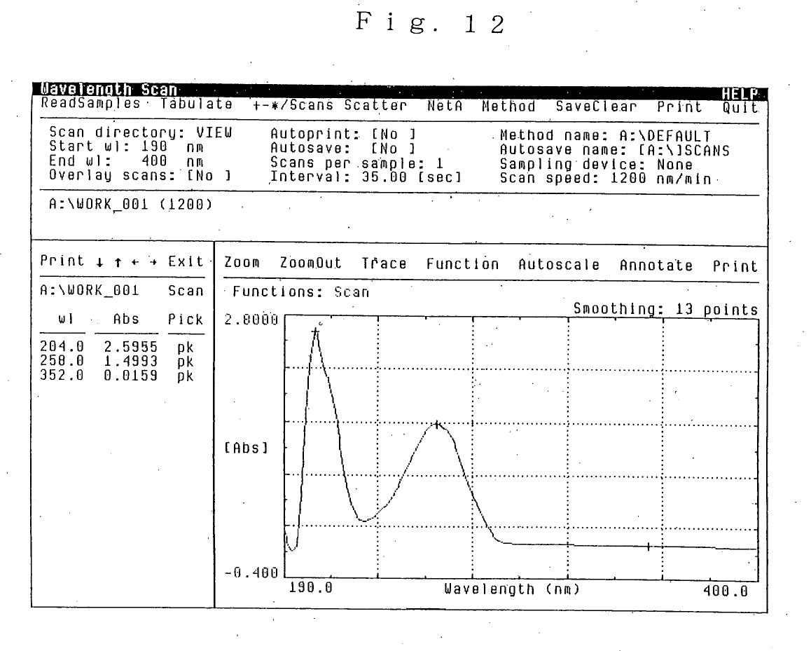

With regard to Compound B obtained by HPLC (dissolved in ethanol), its ultraviolet and visible spectrum was measured (λmax 259 nm). The result is shown in FIG. 12.

From the results described above, Compound B was identified as N-hydroxy-N-methyl-adenosine (the following formula).

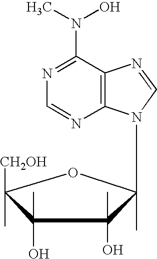

According to the present invention, a histamine release inhibitor containing any of the following <1> to <10> substances as a major component can be obtained.
<1> Adenosine
<2> Guanosine
<3> Cytidine
<4> Uridine
<5> Adenosine monophosphate (AMP)
<6> Adenosine diphosphate (ADP)
<7> Adenosine triphosphate (ATP)
<8> Adenosine derived from Phellinus linteus
<9> N-hydroxy-N-methyl-adenosine
<10> N-hydroxy-N-methyl-adenosine derived from Phellinus linteus

1. A histamine release inhibitor comprising a nucleoside derived from RNA.

2. The histamine release inhibitor according to claim 1, wherein the nucleoside derived from RNA is adenosine.

3. The histamine release inhibitor according to claim 2, wherein adenosine obtained from Phellinus linteus mycelium is used.

4. The histamine release inhibitor according to claim 1, wherein the nucleoside derived from RNA is guanosine.

5. The histamine release inhibitor according to claim 1, wherein the nucleoside derived from RNA is cytidine.

6. The histamine release inhibitor according to claim 1, wherein the nucleoside derived from RNA is uridine.

7. A histamine release inhibitor comprising adenosine monophosphate (AMP).

8. A histamine release inhibitor comprising adenosine diphosphate (ADP).

9. A histamine release inhibitor comprising adenosine triphosphate (ATP).

10. A histamine release inhibitor comprising N-hydroxy-N-methyl-adenosine.

11. The histamine release inhibitor according to claim 10, wherein N-hydroxy-N-methyl-adenosine obtained from Phellinus linteus mycelium is used.
